(19) United States
(12) Patent Application Publication
(10) Pub. No.: US 2023/0022519 A1
(43) Pub. Date: Jan. 26, 2023

(54) THREE-DIMENSIONAL STRUCTURE AND METHOD FOR MANUFACTURING THE SAME

(71) Applicant: NIPPON TELEGRAPH AND TELEPHONE CORPORATION, Tokyo (JP)

(72) Inventors: Koji SAKAI, Musashino-shi, Tokyo (JP); Tetsuhiko TESHIMA, Musashino-shi, Tokyo (JP); Hiroki MIYAZAKO, Musashino-shi, Tokyo (JP); Yuko UENO, Musashino-shi, Tokyo (JP)

(73) Assignee: NIPPON TELEGRAPH AND TELEPHONE CORPORATION, Tokyo (JP)

(21) Appl. No.: 17/787,805

(22) PCT Filed: Dec. 23, 2019
(86) PCT No.: PCT/JP2019/050356

§ 371 Date: Jun. 21, 2022

(51) Int. Cl. C12M 1/12; C12M 1/34; G01N 15/06; B05D 1/00; C23C 16/26; C03C 17/42; G03F 7/16

(52) U.S. Cl. CPC C12M 25/02; C12M 41/36; G01N 15/06; B05D 1/60; C23C 16/26; C03C 17/42; G03F 7/162; G01N 2015/0693; C03C 2218/116; C03C 2218/1525; C03C 2218/33; C03C 2218/355

(57) ABSTRACT

There is provided a three-dimensional structure in which a multilayer film is three-dimensionally curved to form an interior space. The multilayer film includes a layer containing a carbon monoatomic layer substance, a support layer, and a curve induction layer that induces a curved structure, where the layer containing the carbon monoatomic layer substance is in contact with the interior space, and the support layer is positioned between the layer containing the carbon monoatomic layer substance and the curve induction layer.

### TECHNICAL FIELD

The present disclosure relates to three-dimensional structures and manufacturing methods for three-dimensional structures.

### BACKGROUND ART

A monoatomic layer substance constituted with carbon atoms and represented by graphene, has biological compatibility in addition to high electric conductivity, thermal conductivity, and chemical stability. Because of this, there has been an increasing demand in recent years for the monoatomic layer substance as a material for constituting a sensor of a bio-instrumentation device. The scale of biological tissue to be measured by a bio-instrumentation device is in widespread demand from individual levels of humans and animals to cellular levels. Signals to be measured by the bio-instrumentation device widely range from bioelectric signals to biomolecules represented by proteins and amino acids.

As a bio-instrumentation device using graphene, there is an example in which an extracellular potential accompanying firing of nerve cells in the brain is measured using the graphene as an electrode. In addition, there exists an example in which a sensor sensory part made of graphene oxide is modified by molecules capable of detecting biological content (Patent Literature (PTL) 1).

However, because the carbon monoatomic layer has a property of being flat, biomolecules and currents to be measured are likely to diffuse to the surroundings. In addition, because a contact area between a planar sensor portion and cells is small, it is difficult to efficiently measure small amounts of signals emitted by the cells. Furthermore, the sensor portion may not be brought into contact with a three-dimensional biological tissue. In view of the above problems, a method has been studied in which a carbon monoatomic layer is deformed into a variety of shapes to enclose cells by utilizing mechanical strength and mechanical flexibility of the carbon monoatomic layer.

As a method of inducing curvature of a carbon monoatomic layer, a method in which a surface of the carbon monoatomic layer is treated with a polymer is reported (Non Patent Literature (NPL) 1). In addition, a method is reported in which a multilayer thin film is formed by laminating a continuous single-layer graphene and a polymer thin film, and a gradient of rigidity is generated in a thickness direction of the multilayer thin film itself so as to bend the multilayer thin film (NPL 2). As a method of constituting a three-dimensional structure capable of enclosing cells therein, a method is reported in which a multilayer thin film is formed by laminating a silk-fibroin-gel layer and a polymer thin film, and the silk-fibroin-gel layer is swollen so as to bend the multilayer thin film (PTL 2).

### CITATION LIST

### Patent Literature

PTL 1: JP 2013-253825 A

### PTL 2: WO 2017/204235

### Non Patent Literature

NPL 1: W. Xu et al., Ultrathin thermoresponsive self-folding 3D graphene. Sci. Adv., 2017, 3 (10), e1701084.

NPL 2: K. Sakai et al., Graphene-based neuron encapsulation with controlled axonal outgrowth. Nanoscale, 2019, 11 (28), 13249-13259.

### SUMMARY OF THE INVENTION

### Technical Problem

However, in the method of NPL 1, because the carbon monoatomic layer is modified with a polymer, cells are not allowed to make direct contact with the carbon monoatomic layer. In order to control the engraftment of cells to the carbon monoatomic layer, it is desirable not to modify the surface of the carbon monoatomic layer.

In the method of NPL 2, the multilayer thin film is bent with the single-layer graphene being set on the outer side. Due to this, when cells are enclosed in the bent structure, the cells are unable to make contact with the single-layer graphene.
In the method of PTL 1, because the three-dimensional structure does not include a carbon monoatomic layer, action potentials of the cells or biomolecules may not be measured directly.

In light of the above circumstances, an object of the present disclosure is to provide a three-dimensional structure having a three-dimensionally curved structure in which a layer containing a carbon monoatomic layer substance is set on the inner side, and a manufacturing method for the three-dimensional structure.

### Means for Solving the Problem

An aspect of the present disclosure is a three-dimensional structure including a multilayer film that is made into three-dimensionally curving to form an interior space of the three-dimensional structure, in which the multilayer film includes a layer containing a carbon monoatomic layer substance, a support layer, and a curve induction layer that induces a curved structure, the layer containing the carbon monoatomic layer substance is in contact with the interior space, and the support layer is positioned between the layer containing the carbon monoatomic layer substance and the curve induction layer.

An aspect of the present disclosure is a manufacturing method for a three-dimensional structure, the method including (a) forming a multilayer film that includes a layer containing a carbon monoatomic layer substance, a support layer, and a curve induction layer that induces a curved structure, where the support layer is positioned between the layer containing the carbon monoatomic layer substance and the curve induction layer, and (b) causing the multilayer film to form, in a self-organized manner, a three-dimensionally curved shape where the layer containing the carbon monoatomic layer substance is set on an inner side of the three-dimensionally curved shape by utilizing a gradient of strain in a thickness direction of the multilayer film as a drive force.

An aspect of the present disclosure is a laminated body including a substrate, a sacrifice layer laminated on the substrate, a curve induction layer that is laminated on the sacrifice layer and induces a curved structure, a support layer laminated on the curve induction layer, and a layer containing a carbon monoatomic layer substance laminated on the support layer.

### Effects of the Invention

The present disclosure provides a three-dimensional structure having a three-dimensionally curved structure in which a layer containing a carbon monoatomic layer substance is set on the inner side, and a manufacturing method for the three-dimensional structure.

### BRIEF DESCRIPTION OF DRAWINGS

FIG. 1A is a conceptual diagram of a three-dimensional structure according to an aspect of the present disclosure. By curving a multilayer film 1 (left drawing), a three-dimensional structure 100 (right drawing) is formed.

FIG. 1B is a conceptual diagram of a three-dimensional structure according to an aspect of the present disclosure. By curving the multilayer film 1 with a cell 2 being present on a surface thereof (left drawing), the three-dimensional structure 100 (right drawing) enclosing the cell 2 is formed.

FIG. 2A illustrates an example of a configuration of a multilayer film.

FIG. 2B illustrates an example of a configuration of a multilayer film.

FIG. 2C illustrates an example of a configuration of a multilayer film.

FIG. 3 illustrates an example of a three-dimensional structure including a biomolecule detection probe.

FIG. 4 illustrates an example of an electric signal detection device according to an aspect of the present disclosure.

FIG. 5 illustrates an example of an electric signal detection device according to an aspect of the present disclosure.

FIG. 6 is a schematic diagram explaining an example of a manufacturing method for a three-dimensional structure according to an aspect of the present disclosure.

FIG. 7A includes phase-contrast microscopic images showing a process of forming a three-dimensional structure by curvature of a multilayer film in which a layer containing a carbon monoatomic layer substance is single-layer graphene and a curve induction layer is multilayer graphene (two layers). There are respectively shown a phase-contrast microscopic image when 0 seconds have passed, a phase-contrast microscopic image when 10 seconds have passed, and a phase-contrast microscopic image when 27 seconds have passed since the addition of EDTA.

FIG. 7B includes phase-contrast microscopic images showing a process of forming a three-dimensional structure by curvature of a multilayer film in which a layer containing a carbon monoatomic layer substance is a graphene oxide flake and a curve induction layer is single-layer graphene (two layers). There are respectively shown a phase-contrast microscopic image when 0 seconds have passed, a phase-contrast microscopic image when 20 seconds have passed, and a phase-contrast microscopic image when 40 seconds have passed since the addition of EDTA.

FIG. 8A depicts a relationship between the number of layers of graphene in a curve induction layer and a curvature radius of a three-dimensional structure. It may be confirmed that the curvature radius of the three-dimensional structure decreases as the number of layers of graphene in the curve induction layer increases.

FIG. 8B is a graph indicating a relationship among the number of layers of graphene in a curve induction layer, a thickness of a support layer, and a curvature radius of a three-dimensional structure. It may be confirmed that the curvature radius of the three-dimensional structure decreases as the number of layers of graphene in the curve induction layer and the thickness of the support layer increase.

FIG. 9 is a phase-contrast microscopic image of a three-dimensional structure in which a cell is enclosed. Immediately after seeding the cell, a sacrifice layer was dissolved, and the curvature of a multilayer film was induced.

### DESCRIPTION OF EMBODIMENTS

Hereinafter, embodiments of the present disclosure will be described in detail with reference to the drawings in some cases. In the drawings, identical or corresponding portions are assigned the identical or corresponding reference signs, and redundant descriptions thereof will be omitted. Some of the dimensional ratios in the drawings are exaggerated for convenience of explanation, and do not necessarily match the actual dimensional ratios.

### Three-Dimensional Structure

A three-dimensional structure according to an aspect of the present disclosure is a three-dimensional structure in which a multilayer film is three-dimensionally curved to form an interior space. The multilayer film includes a layer containing a carbon monoatomic layer substance, a support layer, and a curve induction layer that induces a curved structure. The layer containing the carbon monoatomic layer substance is in contact with the interior space, and the support layer is positioned between the layer containing the carbon monoatomic layer substance and the curve induction layer. Hereinafter, the drawings indicating a preferred aspect of the present disclosure will be cited, and a three-dimensional structure according to the present aspect will be described.

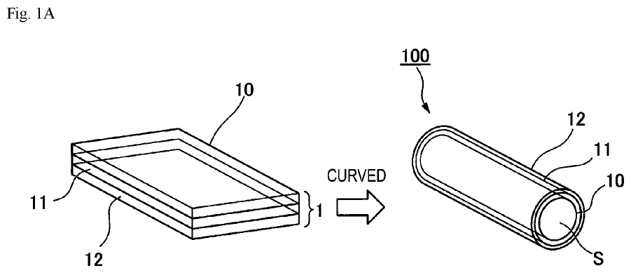

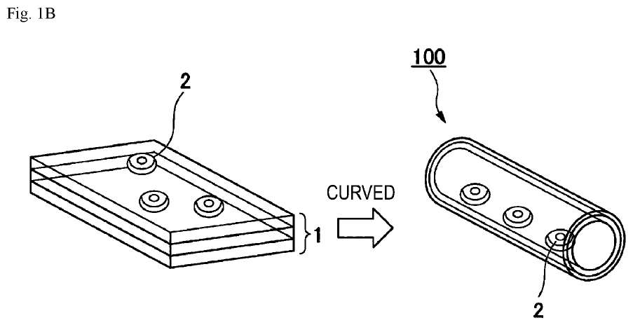

FIG. 1A is a diagram illustrating an example of a three-dimensional structure (right drawing) according to an aspect of the present disclosure and a multilayer film (left drawing) for forming the three-dimensional structure. A three-dimensional structure 100 is formed by a multilayer film 1 being three-dimensionally curved and has an interior space S defined by the multilayer film 1 (right drawing in FIG. 1A). The multilayer film 1 includes a layer 10 containing a carbon monoatomic layer substance, a support layer 11, and a curve induction layer 12 that induces a three-dimensional curve of the multilayer film 1. The support layer 11 is positioned between the layer 10 containing the carbon monoatomic layer substance and the curve induction layer 12 (left drawing in FIG. 1A).

The multilayer film 1 curves with the layer 10 containing the carbon monoatomic layer substance being on the inner side so as to form the three-dimensional structure 100 having the interior space S. Due to this, in the three-dimensional structure 100, the layer 10 containing the carbon monoatomic layer substance is in contact with the interior space S. On the other hand, the curve induction layer 12 is positioned on the outer side and is in contact with an exterior space of the three-dimensional structure 100.

In FIG. 1B, a cell 2 is enclosed in the three-dimensional structure 100. With the cell 2 present on a surface of the layer 10 containing the carbon monoatomic layer substance of the multilayer film 1 (left drawing in FIG. 1B), the cell 2 may be enclosed in the three-dimensional structure 100 by curving the multilayer film 1 (right drawing in FIG. 1B).

In the examples of FIGS. 1A and 1B, the rectangular multilayer film 1 is curved to form the cylindric three-dimensional structure 100, but the shape of the three-dimensional structure is not limited thereto. Changing the thickness and shape of the multilayer film makes it possible to form three-dimensional structures having a variety of three-dimensionally curved shapes. Examples of the three-dimensionally curved shapes include a spherical shape and a spheroidal shape. Furthermore, the cylindric three-dimensional structure is not limited to a structure having a circular cross section, and there may be employed a structure having a cross section formed in an oval shape, a polygonal shape (such as a triangular shape, square shape, pentagonal shape, or hexagonal shape), or the like. The shape of the interior space included in the three-dimensional structure 100 varies in accordance with the three-dimensionally curved shape, and examples thereof include a cylindrical shape, spherical shape, spheroidal shape, polygonal prism shape, polygonal pyramid shape, and circular cone shape.

### Multilayer Film

The multilayer film 1 includes the layer 10 containing the carbon monoatomic layer substance, the support layer 11, and the curve induction layer 12. In the multilayer film 1, the support layer 11 is positioned between the layer 10 containing the carbon monoatomic layer substance and the curve induction layer 12. In other words, the multilayer film 1 is a laminated body in which the curve induction layer 12, the support layer 11, and the layer 10 containing the carbon monoatomic layer substance are laminated in that order.

### Layer Containing Carbon Monoatomic Layer Substance

The layer 10 containing the carbon monoatomic layer substance is a layer containing a carbon monoatomic layer substance. The “carbon monoatomic layer substance” refers to a substance constituted of one layer of a carbon atom layer. The “layer containing the carbon monoatomic layer substance” refers to a layer containing a substance constituted of one layer of a carbon atom layer. The carbon monoatomic layer substance used in the layer 10 containing the carbon monoatomic layer substance is not limited as long as the substance is constituted of one layer of a carbon monoatomic layer. Examples of the carbon monoatomic layer substance include a nanomaterial capable of being processed into a thin film shape (a material at least one dimension of which is not greater than 100 nm). The carbon monoatomic layer substance is preferably a material that does not induce a large volume change when immersed in a solution. The carbon monoatomic layer substance is preferably a material having high light transmittance because such material makes it possible to observe the interior of the three-dimensional structure 100. In the case where the three-dimensional structure 100 encloses cells therein, the carbon monoatomic layer substance is preferably a material having high biological compatibility. In addition, the carbon monoatomic layer substance is preferably a material that performs π-π interaction with a compound contained in the support layer 11. By selecting such carbon monoatomic layer substance, the adhesion between the layer 10 containing the carbon monoatomic layer substance and the support layer 11 may be enhanced.

Examples of the carbon monoatomic layer substance include graphene and graphene derivatives. The carbon monoatomic layer substance contained in the layer 10 containing the carbon monoatomic layer substance may be of one type or may be of two or more types, but is preferably of one type. The layer 10 containing the carbon monoatomic layer substance may be, for example, constituted of graphene or a graphene derivative.

As the carbon monoatomic layer substance, graphene or a graphene derivative is preferable. Examples of graphene derivatives include graphene oxide and graphene oxide reductants. Because of high biological compatibility of graphene and its derivatives, cells may be cultured for a long period of time while being enclosed in the three-dimensional structure 100. In a case where the three-dimensional structure 100 is implanted in a living body, inflammation is unlikely to occur after the implantation. Furthermore, because graphene and derivatives thereof have high transparency, it is also possible to perform evaluation in conjunction with imaging.

When the layer 10 containing the carbon monoatomic layer substance contains graphene as the carbon monoatomic layer substance, the layer 10 containing the carbon monoatomic layer substance may be constituted of single-layer graphene or a plurality of layers of single-layer graphene (hereinafter, also referred to as “multilayer graphene”).

The layer 10 containing the carbon monoatomic layer substance may be constituted of a flake of the carbon monoatomic layer substance. The layer 10 containing the carbon monoatomic layer substance may be constituted of single-layer graphene or a flake of a graphene derivative (graphene oxide, graphene oxide reductant, or the like).
A continuous single-layer graphene has superior conductivity compared to the flake. Because of this, when the layer 10 containing the carbon monoatomic layer substance is used as an electrode or a conductor for detecting an electric signal, single-layer graphene or multilayer graphene is preferably used.
Meanwhile, the flake of a graphene derivative (graphene oxide, graphene oxide reductant, or the like) is able to form the layer 10 containing the carbon monoatomic layer substance simply and inexpensively, and is therefore suitable for measuring biomolecules using an optical molecular probe.

When the layer 10 containing the carbon monoatomic layer substance is constituted of multilayer graphene, the number of layers is not limited, but it is preferable for the layer 10 to be constituted of 1 to 30 layers. From the perspective of maintaining transparency of the multilayer film 1, the layer 10 containing the carbon monoatomic layer substance is more preferably constituted of one to four layers of single-layer graphene.

The thickness of the layer 10 containing the carbon monoatomic layer substance is preferably 0.3 to 10 nm. From the perspective of forming a stable three-dimensionally curved shape, the thickness of the layer 10 containing the carbon monoatomic layer substance is preferably 0.3 to 7 nm, more preferably 0.3 to 5 nm, and even more preferably 0.3 to 1.2 nm.

### Support Layer

The support layer 11 preferably contains a polymer compound that includes many aromatic rings in the molecule and performs π-π interaction with the carbon monoatomic layer substance contained in the layer 10 containing the carbon monoatomic layer substance. By using such a polymer compound, the adhesion of the support layer 11 to the layer 10 containing the carbon monoatomic layer substance is enhanced. The support layer 11 preferably has an insulating property in order to prevent the conduction between the layer 10 containing the carbon monoatomic layer substance and the curve induction layer 12. The support layer 11 is preferably constituted of a material having high light transmittance and high biological compatibility. For example, the support layer 11 may use a polymer compound having high light transmittance and having no cytotoxicity. Examples of such a polymer compound include poly-para-xylene or derivatives thereof. Examples of the derivatives of poly-para-xylene include a polymer of halogenated para-xylene (chloro-para-xylene, fluoro-para-xylene, or the like).

Among them, poly-para-xylene is preferable as the polymer compound to be used in the support layer 11. Poly-para-xylene has high biological compatibility and is highly insulative. Because poly-para-xylene is highly transparent, it is also possible to perform evaluation in conjunction with imaging. In addition, a thin film of poly-para-xylene is flexible and robust. Thus, even a thin film on a nanometer level is able to maintain a three-dimensionally curved structure formed by the multilayer film 1. Furthermore, peeling and tearing from the layer 10 containing the carbon monoatomic layer substance are unlikely to occur, and the desired three-dimensionally curved shape may be formed without losing conductivity.

The polymer compound contained in the support layer 11 may be of one type or may be of two or more types, but is preferably of one type. The support layer 11 may be constituted of a thin film of a polymer compound. The thin film of the polymer compound may be formed with a single layer or a plurality of layers.

The thickness of the support layer 11 is preferably 10 to 900 nm. When the support layer 11 is constituted of a plurality of thin film layers, the total thickness of the plurality of thin film layers is the thickness of the support layer 11. From the perspective of forming a stable three-dimensionally curved shape, the thickness of the support layer 11 is preferably 40 to 400 nm, and more preferably 50 to 250 nm.

### Curve Induction Layer

The curve induction layer 12 is constituted of a material having deformability (hereinafter, also referred to as a “deformation material”). The deformation material is not limited, and any material may be used. The deformation material may be a material having deformability alone or a material having deformability by the interaction with the layer 10 containing the carbon monoatomic layer substance and the support layer 11. Examples of the deformation material include hydrogel (silk-fibroin-gel, gelatin, or the like) that causes a change in volume when being swollen, a polymer thin film having a gradient in cross-linking density, and a continuous monoatomic layer.

The curve induction layer 12 preferably contains a material having adhesion with respect to the support layer 11. For example, in a case where the support layer 11 includes a polymer compound containing an aromatic ring, the curve induction layer 12 preferably includes a substance that performs π-π interaction with the polymer compound. By enhancing the adhesion between the support layer 11 and the curve induction layer 12, the multilayer film 1 may be curved without the support layer 11 and the curve induction layer 12 being peeled from each other.

The curve induction layer 12 is preferably constituted of a material having high biological compatibility. Further, the curve induction layer 12 is preferably constituted of a material having high transparency. By constituting the curve induction layer 12 with a highly transparent material, an optical observation technique may be applied. In particular, when biomolecules are detected using a biomolecule detection probe, it is preferable to use a material having high transmittance in order to optically observe a detection signal of the biomolecule detection probe.

In particular, it is preferable for the curve induction layer 12 to include graphene. Graphene has high biological compatibility and light transmittance. In addition, thickness adjustment is easily made by the number of layers, which makes it easy to control the curvature after the curving. When graphene is used for the curve induction layer 12, the curve induction layer 12 may be constituted of single-layer graphene or multilayer graphene. When the curve induction layer 12 is constituted of graphene, the number of layers is not limited to any specific number. When the layer 10 containing the carbon monoatomic layer substance is also constituted of graphene, the number of layers of graphene in the curve induction layer 12 is preferably greater than the number of layers of graphene in the layer 10 containing the carbon monoatomic layer substance. By increasing the number of layers in the curve induction layer 12, a gradient of stress is generated in the multilayer film 1, so that a curve in which the layer 10 containing the carbon monoatomic layer substance is set on the inner side is likely to be generated. In the case where the layer 10 containing the carbon monoatomic layer substance is constituted of a flake of graphene or a flake of a graphene derivative, the curve induction layer 12 may be constituted of single-layer graphene. In this case, because the stress that contributes to curvature is not generated in the flake of the layer 10 containing the carbon monoatomic layer substance, a gradient of stress is generated in the multilayer film 1, so that the multilayer film 1 may be curved with the layer 10 containing the carbon monoatomic layer substance being on the inner side. In order to maintain the transparency of the multilayer film 1, it is preferable for the curve induction layer 12 to be constituted with one to four layers of graphene.

The thickness of the curve induction layer 12 is preferably 0.3 to 10 nm. From the perspective of forming a stable three-dimensionally curved shape, the thickness of the curve induction layer 12 is preferably 0.3 to 7 nm, more preferably 0.3 to 5 nm, and even more preferably 0.3 to 1.2 nm.

The ratio of the thickness of the layer 10 containing the carbon monoatomic layer substance to the thickness of the support layer 11 (thickness of the layer 10 containing the carbon monoatomic layer substance/thickness of the support layer 11) is preferably in a range from 1/3000 to 1/1, and more preferably in a range from 1/1200 to 1/4. The ratio of the thickness of the curve induction layer 12 to the thickness of the support layer 11 (thickness of the curve induction layer 12/thickness of the support layer 11) is preferably in a range from 1/3000 to 1/1, and more preferably in a range from 1/1200 to 1/4.

When the thickness of the layer 10 containing the carbon monoatomic layer substance is increased, the curvature radius of the three-dimensionally curved shape of the three-dimensional structure 100 may be increased. In contrast, when the thickness of the curve induction layer 12 is increased, the curvature radius of the three-dimensionally curved shape of the three-dimensional structure 100 may be reduced. When the thickness of the support layer 11 is increased, the curvature radius of the three-dimensionally curved shape of the three-dimensional structure 100 may be increased.

Although the multilayer film 1 may or may not include, in addition to the layer 10 containing the carbon monoatomic layer substance, the support layer 11, and the curve induction layer 12, another layer, it is preferable not to include another layer. In other words, the multilayer film 1 is a laminated body constituted of the layer 10 containing the carbon monoatomic layer substance, the support layer 11, and the curve induction layer 12, and it is preferable that the curve induction layer 12, the support layer 11, and the layer 10 containing the carbon monoatomic layer substance are laminated in that order. In the laminated body, the layer 10 containing the carbon monoatomic layer substance is adjacent to the support layer 11, and the support layer 11 is adjacent to the curve induction layer 12.

### Configuration Example of Multilayer Film

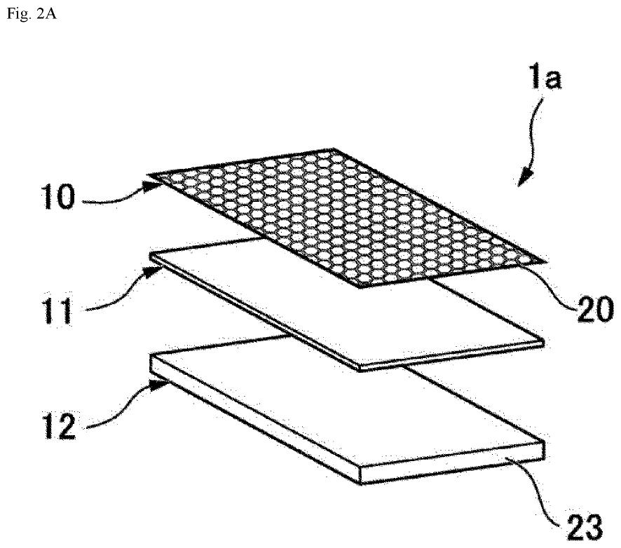

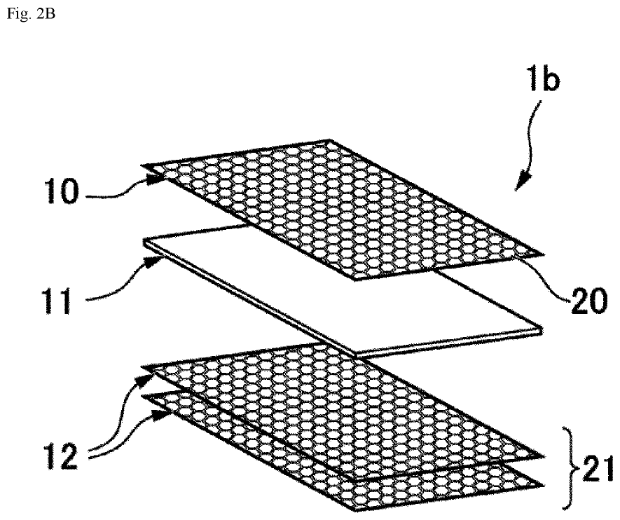

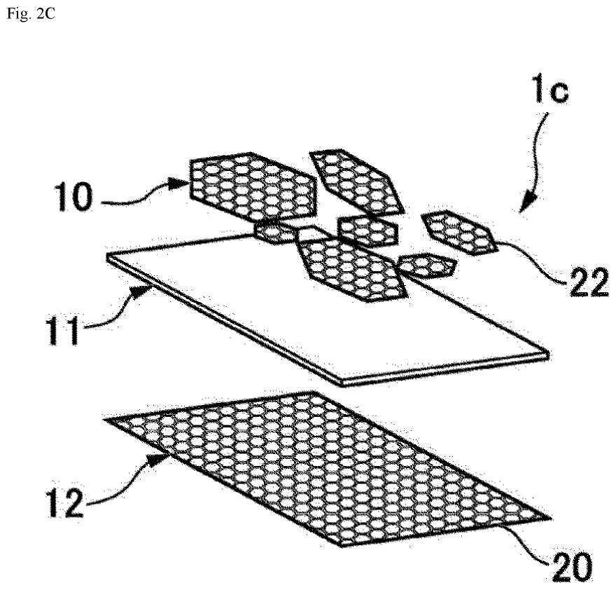

FIGS. 2A to 2C each illustrate a configuration example of a multilayer film.

A multilayer film 1a in FIG. 2A is configured such that a layer 10 containing a carbon monoatomic layer substance is constituted of single-layer graphene 20, and a curve induction layer 12 is constituted of hydrogel 23. For example, silk-fibroin-gel may be used as the hydrogel 23. The hydrogel 23 swells with moisture and changes in volume. On the other hand, the single-layer graphene 20 of the layer 10 containing the carbon monoatomic layer substance does not swell with moisture. Due to this, when the multilayer film 1a is brought into contact with moisture, a stress gradient is generated in the multilayer film 1 by the change in volume of the hydrogel 23. As a result, the multilayer film 1 curves with the layer 10 containing the carbon monoatomic layer substance being on the inner side.

A multilayer film 1b in FIG. 2B is configured such that a layer 10 containing a carbon monoatomic layer substance is constituted of single-layer graphene 20, and a curve induction layer 12 is constituted of multilayer graphene 21. The multilayer graphene 21 is constituted with a plurality of layers of single-layer graphene 20. The multilayer graphene 21 of the curve induction layer 12 has a curving drive force stronger than that of the single-layer graphene 20 of the layer 10 containing the carbon monoatomic layer substance, and thus a stress gradient is generated in the multilayer film 1. As a result, the multilayer film 1 curves with the layer 10 containing the carbon monoatomic layer substance being on the inner side.

A multilayer film 1c in FIG. 2C is configured such that a layer 10 containing a carbon monoatomic layer substance is constituted of a flake 22 of a graphene derivative, and a curve induction layer 12 is constituted of single-layer graphene 20. As the flake 22 of the graphene derivative, for example, a graphene oxide flake may be used. While no stress is generated in the flake 22 of the layer 10 containing the carbon monoatomic layer substance, stress is generated in the single-layer graphene 20 of the curve induction layer 12, so that a stress gradient is generated in the multilayer film 1. As a result, the multilayer film 1 curves with the layer 10 containing the carbon monoatomic layer substance being on the inner side.

In each of FIGS. 2A to 2C, a support layer 11 may be constituted of a polymer compound having a large number of aromatic rings. The support layer 11 is constituted of, for example, poly-para-xylene or a derivative thereof

### Other Configurations

The three-dimensional structure of the present embodiment may have other configurations in addition to the configurations described above.

### Cells

The three-dimensional structure of the present embodiment may allow cells to be present in the interior space S thereof. The expression “to be present in the interior space” means that at least some of the cells are present in the interior space defined by the multilayer film. Not all the cells are included to be present in the interior space S. The three-dimensional structure enclosing the cells may be used as a transplanting tissue to a living body. The three-dimensional structure having been transplanted to the living body may be used to monitor electric activities of the transplanting tissue and the host tissue.

The cells may be animal cells or plant cells, but animal cells are preferred. Mammal cells are preferred as the animal cells. The mammal cells include human cells and cells of mammals other than humans. The cells of mammals other than humans include cells of primates (such as a chimpanzee, gorilla, and monkey), cells of domesticated animals (such as a bovine, porcine, ovine, and equine), cells of rodents (such as a mouse, rat, guinea pig, and hamster), and cells of pets (such as a dog, cat, and rabbit).

Cell types of the cells are not limited, and any cell within the living body may be cited. Examples of the cells 2 include nerve cells, glial cells, cardiomyocytes, fibroblasts, and vascular epithelial cells. When the three-dimensional structure serves as a nerve tissue for transplantation, examples of the cells 2 include nerve cells and glial cells. The cell may be one type of cell or may be a mixture of multiple types of cells. The mixture of cells includes, for example, a mixture of a nerve cell and a glial cell.

The number of cells enclosed in the interior space S of the three-dimensional structure is not limited. The number of cells may be any number corresponding to the size of the interior space S. The cells may be cultured while being enclosed in the three-dimensional structure, and multiplied in the interior space S. Thus, regardless of the number of cells initially enclosed, by culturing the cells continuously, the interior space S may be filled with an appropriate number of cells.

When the three-dimensional structure encloses cells, the cells are preferably in contact with the layer containing the carbon monoatomic layer substance. In the three-dimensional structure of the present embodiment, the layer containing the carbon monoatomic layer substance is disposed on the inner side, whereby the cell present in the interior space S is able to make contact with the layer containing the carbon monoatomic layer substance. The contact of the cell with the layer containing the carbon monoatomic layer substance makes it possible to detect an electric signal generated from the cell in a highly sensitive manner. Furthermore, because the three-dimensional structure has a three-dimensionally curved structure, the area of the layer containing the carbon monoatomic layer substance in contact with the cell may be increased compared to a case where the layer containing the carbon monoatomic layer substance has a planar shape.

### Pore

The three-dimensional structure of the present embodiment may have a pore that makes the interior space S communicate with the exterior space. In the case where the three-dimensional structure has a pore, the pore is formed in the multilayer film. By the multilayer film having the pore, it is possible to exchange substances between the interior space and the exterior space of the three-dimensional structure. Thus, when cells are enclosed in the three-dimensional structure, the permeability of nutrients and oxygen is improved, and the growing environment for the cells is made favorable.

In the case where the multilayer film has a pore, it is preferable that the pore diameter of the pore be smaller than the cell 2 in order to prevent the cells from flowing out through the pore. The shape of the pore is not limited and may take any shape. Examples of the cross-sectional shape of the pore include, but are not limited to, circular, oval, and polygonal (triangular, square, hexagonal, and the like) shapes. In view of ease of the formation or the like, the cross-sectional shape of the pore is preferably circular or oval.

### Biomolecule Detection Probe

The three-dimensional structure may include a biomolecule detection probe. The biomolecule detection probe has a function of changing signal intensity in accordance with the concentration of biomolecules. The biomolecule detection probe may be used for detection of biomolecules secreted from cells, and the like. Examples of the biomolecule detection probe include a probe described in JP 2013-253825 A.

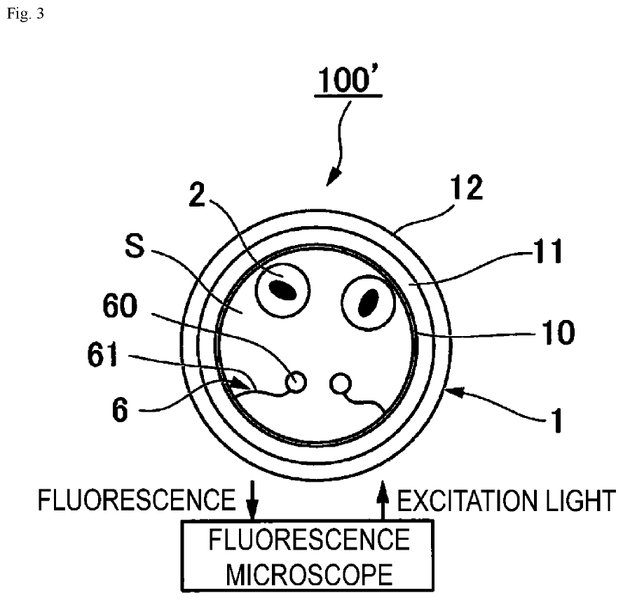

FIG. 3 is a diagram illustrating an example of a three-dimensional structure including a biomolecule detection probe. A three-dimensional structure 100′ is formed in such a manner that a multilayer film 1 including a layer 10 containing a carbon monoatomic layer substance, a support layer 11, and a curve induction layer 12 is three-dimensionally curved. The three-dimensional structure 100′ encloses a cell 2 in an interior space S. The layer 10 containing the carbon monoatomic layer substance includes a biomolecule detection probe 6.

The biomolecule detection probe 6 is constituted of a specific binding substance 61 that specifically binds with a biomolecule to be detected, and a signal molecule 60.

The specific binding substance 61 is not limited to any specific substance, as long as it is a substance capable of specifically binding with a biomolecule to be detected. As the specific binding substance 61, an aptamer is cited, for example. The aptamer is a nucleic acid (nucleic acid aptamer) or peptide (peptide aptamer) having a function to specifically bind with a specific molecule. The aptamer used for the specific binding substance 61 may be a nucleic acid aptamer or peptide aptamer. Examples of the nucleic acid aptamer include an RNA aptamer, a DNA aptamer, and an RNA/DNA aptamer formed of RNA and DNA. The nucleic acid aptamer may contain artificial nucleic acids (BNA, LNA, PNA, and the like). Aptamers that binds with a target molecule may be selected by a known method such as the SELEX method or the Two-hybrid method. The binding target of the specific binding substance 61 is not limited, and any biomolecule may be the target. Examples of the binding target include, but are not limited to, various proteins, enzymes, antibodies, receptors, hormones, peptide, lipid, amino acids, and antibiotics.

The signal molecule 60 is a molecule that generates a signal such as fluorescence. The signal generated by the signal molecule 60 is quenched by the layer 10 containing the carbon monoatomic layer substance. The signal quenching ability of the layer 10 containing the carbon monoatomic layer substance varies in accordance with the distance between the signal molecule 60 and the layer 10 containing the carbon monoatomic layer substance. When the distance between the signal molecule 60 and the layer 10 containing the carbon monoatomic layer substance is short, quenching efficiency increases and signal intensity decreases. On the other hand, when the distance between the signal molecule 60 and the layer 10 containing the carbon monoatomic layer substance is long, the quenching efficiency decreases and the signal intensity increases. As the signal molecule 60, a fluorescent molecule is cited. Examples of the fluorescent molecule include, but are not limited to, FAM and FITC.

The biomolecule detection probe 6 is preferably made to adhere to the layer 10 containing a carbon monoatomic layer substance. For example, the signal molecule 60 may be bound to one end of the specific binding substance 61 and the other end of the specific binding substance 61 may be made to adhere to the layer 10 containing the carbon monoatomic layer substance. An adhering method is not limited to any specific method, and a known method may be used. Examples of the adhering method include a method using an adhesion molecule such as an adhesion molecule having an N-hydroxysuccinimide ester site (JP 2013-253825 A).

In the three-dimensional structure 100′, when a biomolecule released by the cell 2 binds with the specific binding substance 61, the structure of the specific binding substance 61 changes. With this, the distance between the signal molecule 60 and the layer 10 containing the carbon monoatomic layer substance changes, and the signal intensity changes. In the case where the signal molecule 60 is a fluorescent molecule, the change in signal intensity may be detected with a fluorescence microscope.

In the three-dimensional structure 100′, it is sufficient for the layer 10 containing the carbon monoatomic layer substance to have an ability of quenching a signal generated by the signal molecule 60. Accordingly, a flake of graphene or a flake of a graphene derivative (graphene oxide, graphene oxide reductant, or the like) may be used from the perspective of being easily and inexpensively manufactured.

The three-dimensional structure of the present embodiment curves with the layer containing the carbon monoatomic layer substance being on the inner side, and the layer containing the carbon monoatomic layer substance is in contact with the interior space. At the time of curving, it is unnecessary to chemically modify the surface of the layer containing the carbon monoatomic layer substance. Thus, the cells may be cultured in the interior space of the three-dimensional structure in a state in which the cells are in contact with the layer containing the carbon monoatomic layer substance. With this, biological signals from the cells enclosed in the interior space may be easily measured.

Because the three-dimensional structure of the present embodiment has a three-dimensionally curved structure, the contact area with the cells or biomolecules is increased compared to a case where the layer containing the carbon monoatomic layer substance has a planar shape. As a result, amplification of the biological signals of the cells or currents derived from the biomolecules is expected.
Furthermore, it is possible for the three-dimensional structure of the present embodiment to measure the biological signals from a three-dimensionally shaped cell aggregation tissue.
According to the three-dimensional structure of the present embodiment, it is possible to measure extracellular potential fluctuations accompanying the firing of the cells by using a carbon monoatomic layer substance such as graphene as an electrode. In addition, it is possible to efficiently measure the biomolecules by making the biomolecule detection probe adhere to the layer containing the carbon monoatomic layer substance (preferably constituted of a flake of graphene oxide or a flake of a graphene oxide reductant).

### Electric Signal Detection Device

An electric signal detection device according to an aspect of the present disclosure includes the three-dimensional structure according to the above aspect, and a conductor connected to the layer containing the carbon monoatomic layer substance of the three-dimensional structure mentioned above.

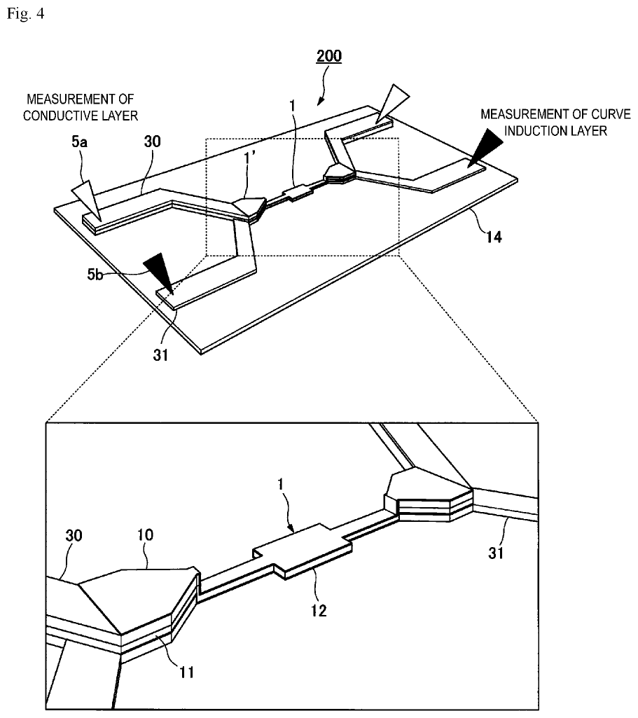

FIG. 4 is a diagram illustrating an example of an electric signal detection device according to the present embodiment. FIG. 4 illustrates a state of a multilayer film 1 before a three-dimensional structure 100 is formed. By generating a gradient of strain in the thickness direction in the multilayer film 1, it is possible to cause the multilayer film 1 to form the three-dimensional structure 100 by utilizing the gradient of strain as a drive force. This makes it possible to obtain an electric signal detection device including the three-dimensional structure 100.

An electric signal detection device 200 includes the multilayer film 1 before forming the three-dimensional structure 100, conductors 30 and 31, a conductor connecting portion 1′, where the conductors 30 and 31 are connected to the multilayer film 1, and a substrate 14, on which these members are mounted. Examples of the material of the substrate 14 include the same materials as those cited in “Manufacturing Method for Three-Dimensional Structure” to be described below.

At the conductor connecting portion 1′, the conductor 31, a curve induction layer 12, a support layer 11, the conductor 30, and the conductor 31 are laminated in that order on the substrate 14. The conductor 30 is connected to a layer 10 containing a carbon monoatomic layer substance of the three-dimensional structure 100 (the multilayer film 1 before curving) via the conductor connecting portion 1′. Because of this, by connecting a probe 5a of a measurement device to the conductor 30, it is possible to measure electric characteristics of the layer 10 containing the carbon monoatomic layer substance. The conductor 31 is connected to the curve induction layer 12 of the three-dimensional structure 100 (the multilayer film 1 before curving) via the conductor connecting portion 1′. Because of this, in a case where the curve induction layer 12 contains a conductive material, by connecting a probe 5b of the measurement device to the conductor 31, it is possible to measure electric characteristics of the curve induction layer 12. In a case where the curve induction layer 12 does not contain a conductive material or in a case where it is unnecessary to measure the electric characteristics of the curve induction layer 12, the conductor 31 may not be provided.

By using a highly insulative material for the support layer 11, the electric characteristics of the layer 10 containing the carbon monoatomic layer substance and the curve induction layer 12 may be independently measured without crosstalk between the conductors 30 and 31. Examples of the material constituting the support layer 11 include poly-para-xylene.

The electric signal detection device 200 may be produced by laminating the conductor 31, the curve induction layer 12, the support layer 11, the conductor 30, and the layer 10 containing the carbon monoatomic layer substance in that order on the substrate 14. While forming the above-mentioned layers, each layer may be patterned in any shape using the photolithography technique or the like. This may allow the multilayer film 1 for forming the three-dimensional structure 100 to have any shape and take any arrangement. Further, the conductors 30 and 31 as well as the conductor connecting portion 1′ may have any shape and take any arrangement.

After the laminated body is produced, the multilayer film 1 is curved to form the three-dimensional structure 100 by generating a stress distribution in the thickness direction in the multilayer film 1. At this time, by measuring resistance values of the layer 10 containing the carbon monoatomic layer substance and the curve induction layer 12 before and after the curving, changes in electric characteristics due to the deformation of the multilayer film 1 may be evaluated.

### Another Embodiment

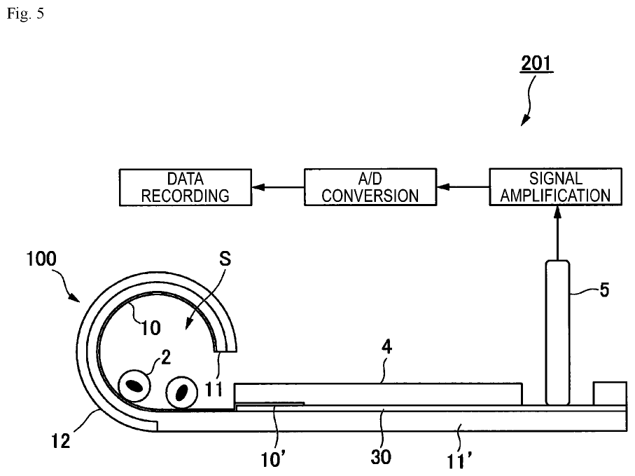

FIG. 5 is a diagram illustrating another embodiment of an electric signal detection device. An electric signal detection device 201 in FIG. 5 includes a three-dimensional structure 100, a conductor 30 connected to a layer 10 containing a carbon monoatomic layer substance of the three-dimensional structure 100, a conductor connecting portion 10′, where the conductor 30 is connected to the layer 10 containing the carbon monoatomic layer substance, a support layer extension 11′ formed by extending a support layer 11 of the three-dimensional structure 100, and an insulating layer 4. In an interior space S of the three-dimensional structure 100, a cell 2 is present.

In the electric signal detection device 201, the conductor 30 is formed on the support layer extension 11′ formed by extending the support layer 11 of the three-dimensional structure 100. The conductor 30 is connected to the layer 10 containing the carbon monoatomic layer substance via the conductor connecting portion 10′ formed by extending the layer 10 containing the carbon monoatomic layer substance of the three-dimensional structure 100. By connecting a probe 5 of a measurement device to the conductor 30, it is possible to measure electric characteristics of the layer 10 containing the carbon monoatomic layer substance. It is also possible to detect a change in electric characteristics caused by the activity of the cell 2.

The conductor 30 is covered with the insulating layer 4. By forming the insulating layer 4, leakage of a current from the conductor 30 may be prevented. At a site where the conductor 30 connects with the probe 5, the insulating layer 4 is not present, and the conductor 30 is preferably exposed.

The electric signal detection device 201 may be produced by laminating a curve induction layer 12, the support layer 11, a conductor 31, the layer 10 containing the carbon monoatomic layer substance, and the insulating layer 4 in that order. While forming the above-mentioned layers, each layer may be patterned in any shape using the photolithography technique or the like. This may allow the multilayer film 1 for forming the three-dimensional structure 100 to have any shape and take any arrangement. Further, the conductor 30, the conductor connecting portion 10′, the support layer extension 11′, and the insulating layer 4 may have any shape and take any arrangement. After the laminated body is produced, the multilayer film 1 is curved to form the three-dimensional structure 100 by generating a stress distribution in the thickness direction in the multilayer film 1. At this time, by making the cell 2 present on the layer 10 containing the carbon monoatomic layer substance and making the multilayer film 1 curved, the cell 2 may be enclosed in the three-dimensional structure 100.

In the electric signal detection devices 200 and 201, the conductors 30 and 31 and the insulating layer 4 may be constituted as follows.

### Conductors

The conductors 30 and 31 are used to connect the layer 10 containing the carbon monoatomic layer substance or the curve induction layer 12, with the measurement device for measuring electric characteristics. The material of the conductors 30 and 31 is not limited to any specific one, as long as the material has conductivity. A material that can be deposited on the support layer 11 is preferable as the material of the conductors 30 and 31. Examples of such material include metals and alloys. A material on which an oxide film is unlikely to be formed is preferable, even when the cell 2 is cultured for a long period of time in the interior space S of the three-dimensional structure 100. In the case where optical observation of the cell 2 is performed, it is preferable for the conductors 30 and 31 to be transparent.
Specific examples of the metals that may be used as the material of the conductors 30 and 31 include gold, copper, platinum, aluminum, titanium, and chromium. Specific examples of the alloys include indium tin oxide.

The thickness of the conductors 30 and 31 is not limited to any specific one but is preferably about 10 to 1000 nm. Specific examples of the conductors 30 and 31 include, but are not limited to, a gold conductor having a thickness of about 50 nm and an indium tin oxide conductor having a thickness of about 300 nm.

### Insulating Layer

The insulating layer 4 is provided for preventing leakage of a current from the conductor 30. It is sufficient for the insulating layer 4 to be constituted of an insulative material, and the quality of material is not limited to any specific one. The material of the insulating layer 4 is preferably a material having high biological compatibility and a high cell engraftment rate. In the case where the optical observation of the cell 2 is performed, it is preferable for the insulating layer 4 to be transparent.
Examples of the material of the insulating layer 4 include polymer compounds such as photoresists. Specific examples of the polymer compounds include photoresists such as OFPR-800 (manufactured by Tokyo Ohka Kogyo Co., Ltd.) and SU-8 (manufactured by Nippon Kayaku Co., Ltd.), poly-para-xylene, and polyimide.

The thickness of the insulating layer 4 is not limited to any specific one but is preferably about 0.1 to 20 μm. Specific examples of the insulating layer 4 include a polymer compound layer having a thickness of about 2 μm and constituted of the polymer compound as described above.

The electric signal detection device of the present embodiment includes the three-dimensional structure and the conductor connected to the layer containing the carbon monoatomic layer substance of the three-dimensional structure, thereby making it possible to directly measure the electric characteristics of the interior space of the three-dimensional structure. When the three-dimensional structure encloses cells therein, the cells may make contact with the layer containing the carbon monoatomic layer substance, which makes it possible to detect the action potential of the cells in a highly sensitive manner.

### Manufacturing Method for Three-Dimensional Structure

A manufacturing method for a three-dimensional structure according to an aspect of the present disclosure includes (a) forming a multilayer film that includes a layer containing a carbon monoatomic layer substance, a support layer, and a curve induction layer that induces a curved structure, where the support layer is positioned between the layer containing the carbon monoatomic layer substance and the curve induction layer, and (b) causing the multilayer film to form, in a self-organized manner, a three-dimensionally curved shape where the layer containing the carbon monoatomic layer substance is set on an inner side of the three-dimensionally curved shape by utilizing a gradient of strain in a thickness direction of the multilayer film as a drive force. The three-dimensional structure obtained by the manufacturing method of the present embodiment includes an interior space formed by the three-dimensionally curved shape of the multilayer film, and the layer containing the carbon monoatomic layer substance is in contact with the interior space. Accordingly, the three-dimensional structure of the embodiment described above may be obtained by the manufacturing method of the present embodiment.

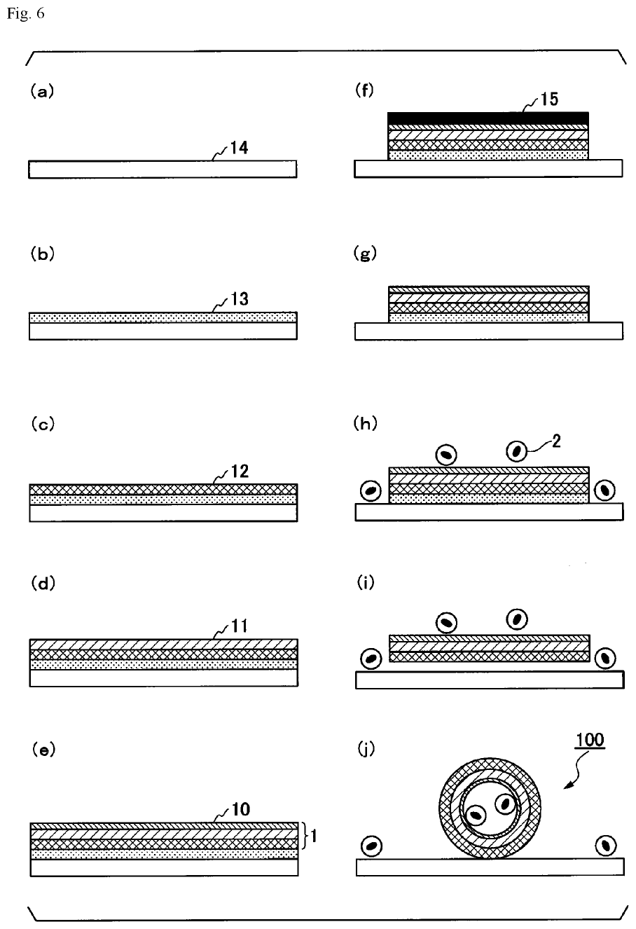

FIG. 6 is a diagram schematically illustrating a manufacturing method for a three-dimensional structure.

First, a multilayer film 1 including a layer 10 containing a carbon monoatomic layer substance, a support layer 11, and a curve induction layer 12 is formed (FIGS. 6(a) to 6(e): step (a)). In an example of FIGS. 6(a) to 6(e), a sacrifice layer 13 is formed on a substrate 14, and then the curve induction layer 12, the support layer 11, and the layer 10 containing the carbon monoatomic layer substance are sequentially laminated on the sacrifice layer 13 to form the multilayer film 1. Subsequently, patterning (FIGS. 6(f) to 6(g)), seeding of cells 2 (FIGS. 6(h) to 6(i))), and the like are performed.
Subsequently, by utilizing a gradient of strain in a thickness direction of the multilayer film 1 as a drive force, the multilayer film 1 is made to form, in a self-organized manner, a three-dimensionally curved shape where the layer 10 containing the carbon monoatomic layer substance is set on an inner side (FIGS. 6(i) to 6(j): step (b)). In an example of FIGS. 6(i) to 6(j), a stress distribution is formed in the thickness direction of the multilayer film 1 by the multilayer film 1 including the curve induction layer 12 having deformability. Accordingly, by dissolving the sacrifice layer 13 and releasing the multilayer film 1 from the substrate 14, a gradient of strain is formed in an in-plane direction of the multilayer film 1 (FIG. 6(i)). The multilayer film 1 bends with the layer 10 containing the carbon monoatomic layer substance being on the inner side by utilizing the gradient of strain as a drive force (FIG. 6(i)), and the three-dimensionally curved shape in which the layer 10 containing the carbon monoatomic layer substance is set on the inner side is built in a self-organized manner (FIG. 6(j)). As a result, a three-dimensional structure 100 may be obtained.
Hereinafter, steps of the manufacturing method for electrodes according to the present aspect will be described.

### Step (a)

Step (a) is a step of forming a multilayer film that includes a layer containing a carbon monoatomic layer substance, a support layer, and a curve induction layer that induces a curved structure, where the support layer is positioned between the layer containing the carbon monoatomic layer substance and the curve induction layer.

The multilayer film 1 may be formed by laminating the curve induction layer 12, the support layer 11, and the layer 10 containing the carbon monoatomic layer substance in that order. The method of forming the multilayer film 1 is not limited, and examples thereof include a method using the substrate 14 and the sacrifice layer 13. In the example of FIGS. 6(a) to 6(g), the sacrifice layer 13 is formed on the substrate 14 (FIGS. 6(a) to 6(b)), the curve induction layer 12 is subsequently formed on the sacrifice layer 13 (FIG. 6(c)), the support layer 11 is subsequently formed on the curve induction layer 12 (FIG. 6(d)), and then the layer 10 containing the carbon monoatomic layer substance is formed on the support layer 11 (FIG. 6(e)), so as to form the multilayer film 1. By forming the multilayer film 1 on the substrate 14 and the sacrifice layer 13, the multilayer film 1 may be formed while maintaining a two-dimensional planar structure.

### Substrate

The substrate 14 is used for convenience of forming the multilayer film 1, and the material thereof is not limited. A material having high surface flatness is preferable as the material of the substrate 14. In the case where, after the manufacture of the three-dimensional structure 100, the cell 2 is observed with a fluorescence microscope or the like while holding the three-dimensional structure 100 on the substrate 14, it is preferable for the substrate 14 to be a member that does not obstruct the observation with the fluorescence microscope.
Examples of the material of the substrate 14 include silicon, soda glass, quartz, magnesium oxide, and sapphire.

The thickness of the substrate 14 is not limited but is preferably about 50 to 200 μm. Specific examples of the substrate 14 include a glass substrate having a thickness of about 100 μm.

### Sacrifice Layer

The sacrifice layer 13 serves as a temporary adhesive layer for releasing, from the substrate 14, the multilayer film 1 including the layer 10 containing the carbon monoatomic layer substance, the support layer 11, and the curve induction layer 12. The sacrifice layer 13 is constituted of a material having characteristics of being dissolved in response to external stimuli by chemicals, temperature changes, light irradiation, and the like. The material of the sacrifice layer 13 is not limited as long as the material is dissolved in response to the external stimulus.
As the sacrifice layer 13, for example, calcium alginate gel, which is a type of physical gel, may be used. The calcium alginate gel transitions from gel to sol and dissolves when an enzyme such as arginase or a chelating agent is added. Examples of the chelating agent that can be used to dissolve the calcium alginate gel include sodium citrate and ethylenediaminetetraacetic acid (EDTA). The enzyme and chelating agent as discussed above are not toxic to biological samples such as cells. Because of this, with the cells 2 present on the layer 10 containing the carbon monoatomic layer substance, these reagents may be added to dissolve the sacrifice layer 13. This allows the interior space of the three-dimensional structure 100 to enclose the cells 2 simply and efficiently.

The thickness of the sacrifice layer 13 is not limited to any specific one. The thickness of the sacrifice layer 13 may be 20 to 1000 nm, for example, in terms of quick dissolution. The method of forming the sacrifice layer 13 on the substrate 14 is not limited, and in accordance with the material of the sacrifice layer 13, a method commonly used for thin film formation may be selected as appropriate. Examples of the method of forming the sacrifice layer 13 include chemical vapor deposition (CVD), spin coating, ink jet printing, vapor deposition, and electrospray.

### Curve Induction Layer, Support Layer, and Layer Containing Carbon Monoatomic Layer Substance

The curve induction layer 12, the support layer 11, and the layer 10 containing the carbon monoatomic layer substance are the same as those described in the section “Three-Dimensional Structure”.

The method of forming the curve induction layer 12 is not limited, and methods such as a transfer method using a water surface, chemical vapor deposition (CVD), spin coating, ink jet printing, thermal vapor deposition, and electrospray are available. When the curve induction layer 12 is constituted of hydrogel, the methods described above may be appropriately selected in accordance with the type of hydrogel.

When the curve induction layer 12 is constituted of single-layer graphene, a method as follows may be used, for example. First, single-layer graphene is formed on a surface of a metal film such as copper foil by using CVD. Subsequently, the metal film is dissolved and washing on the water surface is repeated, and thereafter the single-layer graphene is transferred to the surface of the sacrifice layer 13. As a result, the curve induction layer 12 made of single-layer graphene may be formed. By repeating the operations described above, the curve induction layer 12 made of multilayer graphene may be formed.

The method of forming the support layer 11 is not limited, and the methods such as CVD, spin coating, ink jet printing, vapor deposition, and electrospray are available. For example, in the case where the support layer 11 is constituted of poly-para-xylene or a derivative thereof, by growing para-xylene or a dimer, which is a derivative thereof, by CVD, the support layer 11 may be formed.

The method of forming the layer 10 containing the carbon monoatomic layer substance is not limited, and methods such as a transfer method using a water surface, chemical vapor deposition (CVD), spin coating, ink jet printing, thermal vapor deposition, and electrospray are available. When the layer 10 containing the carbon monoatomic layer substance is constituted of graphene, the layer 10 containing the carbon monoatomic layer substance may be formed by forming single-layer graphene and transferring it to the surface of the support layer 11 by the same method as described in the curve induction layer 12.

When the layer 10 containing the carbon monoatomic layer substance is constituted of a flake of a graphene derivative (graphene oxide, graphene oxide reductant, or the like), the layer 10 containing the carbon monoatomic layer substance may be formed by applying a dispersion liquid containing the flake of the graphene derivative to the support layer 11. The method of applying the dispersion liquid is not limited, and examples thereof include spin coating and casting. The dispersion liquid of the graphene derivative flake may be produced by known methods such as those described in JP 2013-254825 A and the like.

### Step (b)

Step (b) is a step of causing the multilayer film to form, in a self-organized manner, a three-dimensionally curved shape where the layer containing the carbon monoatomic layer substance is set on the inner side by utilizing a gradient of strain in the thickness direction of the multilayer film as a drive force.

The gradient of strain in the thickness direction of the film may be obtained by the laminated structure of the layer 10 containing the carbon monoatomic layer substance, the support layer 11, and the curve induction layer 12. In the example of FIG. 6, the gradient of strain is generated in the thickness direction of the multilayer film 1 by forming the multilayer film 1 including the layer 10 containing the carbon monoatomic layer substance, the support layer 11, and the curve induction layer 12 on the sacrifice layer 13. By dissolving the sacrifice layer 13 (FIG. 6(i)), it is possible to cause the multilayer film 1 to form the three-dimensionally curved shape in the self-organized manner, where the layer 10 containing the carbon monoatomic layer substance is set on the inner side by utilizing the gradient of strain as a drive force (FIG. 6(j)). As a result, the three-dimensional structure 100, in which the layer 10 containing the carbon monoatomic layer substance is in contact with the interior space of the three-dimensionally curved shape, may be obtained.

The sacrifice layer 13 may be dissolved as appropriate in accordance with the material of the sacrifice layer 13. For example, in the case where the sacrifice layer 13 is constituted of calcium alginate gel, the sacrifice layer 13 may be dissolved by adding the chelating agent, arginase, or the like as discussed above.

In an embodiment, a manufacturing method according to the present aspect may include forming a sacrifice layer on a substrate, forming, on the sacrifice layer, a multilayer film in which a curve induction layer, a support layer, and a layer containing a carbon monoatomic layer substance are laminated in that order, and causing the multilayer film to form, in a self-organized manner, a three-dimensionally curved shape where the layer containing the carbon monoatomic layer substance is set on an inner side of the three-dimensionally curved shape by dissolving the sacrifice layer and utilizing a gradient of strain in a thickness direction of the multilayer film as a drive force.

### Other Steps

The manufacturing method of the present embodiment may include other steps in addition to the above-discussed steps (a) and (b). Examples of the other steps include a step of causing a cell to be present, a step of patterning the multilayer film 1, and a step of culturing the cell.

### Step of Causing Cells to Be Present

The manufacturing method according to the present aspect may include a step of causing a cell to be present on the surface of the layer containing the carbon monoatomic layer substance, between the above-discussed steps (a) and (b).

FIG. 6(h) is a schematic diagram illustrating a state in which the cells 2 are present on the surface of the layer 10 containing the carbon monoatomic layer substance. It is sufficient that the cells 2 are present at any position on a vertical position relative to the surface of the layer 10 containing the carbon monoatomic layer substance. The cells 2 may float over the surface of the layer 10 containing the carbon monoatomic layer substance or may adhere to the surface of the layer 10 containing the carbon monoatomic layer substance. In order to enclose the cells 2 in the interior space of the three-dimensional structure 100, which is built in a self-organized manner by the above-discussed step (b), the distance from the surface of the layer 10 containing the carbon monoatomic layer substance to the cell 2 is preferably less than half the length in a short axis direction of the layer 10 containing the carbon monoatomic layer substance.

The method of causing the cells 2 to be present on the surface of the layer 10 containing the carbon monoatomic layer substance is not limited, and any method may be used cooperatively. Examples of the method of causing the cells 2 to be present on the surface of the layer 10 containing the carbon monoatomic layer substance include a method of dropping a culture solution or a suspension of the cells 2 onto the surface of the layer 10 containing the carbon monoatomic layer substance, and a method of immersing the layer 10 containing the carbon monoatomic layer substance in a culture solution or a suspension of the cells 2. The number of cells 2 present on the surface of the layer 10 containing the carbon monoatomic layer substance may be controlled by the concentration of the cells 2 in the culture solution or suspension of the cells 2.

### Patterning Step

The manufacturing method of the present aspect may include a step of patterning the multilayer film 1. The patterning step is preferably performed, after the above-discussed step (a), before the above-discussed step (b). The method of patterning the multilayer film 1 is not limited, and a known patterning method may be used. As the patterning method, a fine processing technique such as a photolithography method, an electron beam lithography method, and a dry etching method is applicable.

FIGS. 6(f) to 6(g) are diagrams illustrating an example of the patterning step. In FIGS. 6(f) to 6(g), the multilayer film 1 is patterned using a resist layer 15. FIG. 6(f) illustrates a state in which the resist layer 15 is formed on the multilayer film 1. The method of forming the resist layer 15 is not limited, and a known method such as spin coating may be used. The resist layer 15 is exposed through a photomask having any shape and is developed using a developing solution, thereby making it possible to obtain a resist pattern of any shape. By etching the multilayer film 1 and the sacrifice layer 13 by using the resist pattern as a physical mask, the multilayer film 1 patterned in any shape may be obtained (FIG. 6(g)). The sacrifice layer 13 may or may not be patterned along with the multilayer film 1. From the perspective of ease of patterning and dissolution performance of the sacrifice layer 13, it is preferable for the sacrifice layer 13 to be patterned along with the layer 10 containing the carbon monoatomic layer substance.

By forming the multilayer film 1 on the substrate 14 and the sacrifice layer 13, the multilayer film 1 is maintained in a two-dimensional planar shape. This makes it possible to perform fine-structured patterning on the multilayer film 1.

The pattern shape formed by patterning is not limited to any specific shape. For example, when obtaining a cylindric three-dimensional structure, the multilayer film 1 is preferably patterned in a rectangular shape. The size of the rectangle may be selected as appropriate in accordance with the size of the cell to be enclosed, the purpose of use of the three-dimensional structure, and the like, and may be, for example, 400 to 4000 μm in length and 20 to 400 μm in width. In addition, pores may be formed in the multilayer film 1 by patterning.

### Culturing Step

The manufacturing method of the present aspect may include a step of culturing a cell. The culturing step may be performed after the step of causing the cell to be present. The culturing step may be performed before step (b) or after step (b). In the case where the cell 2 is a cell having an adhesion property, it is possible to make the cell adhere to the multilayer film 1 by performing the culturing step before step (b). Accordingly, when the multilayer film 1 is made to form a three-dimensionally curved shape in step (b), the cell may be reliably enclosed in the interior space of the three-dimensionally curved shape.
By performing the culturing step after step (b), it is possible to multiply the cells along the three-dimensionally curved shape of the three-dimensional structure 100. In accordance with the type of the cells, commonly used conditions for culturing the cells may be employed as the culturing conditions of the cells.

According to the method of the present aspect, it is possible to obtain the three-dimensional structure of the embodiment described above by a simple method.

### Laminated Body

A laminated body according to an aspect of the present disclosure includes a substrate, a sacrifice layer laminated on the substrate, a curve induction layer laminated on the sacrifice layer, a support layer laminated on the curve induction layer, and a layer laminated on the support layer and containing a carbon monoatomic layer substance.

Specific examples of the laminated body according to the present aspect include a laminated body exemplified in FIG. 6(g).

The substrate 14, the sacrifice layer 13, the curve induction layer 12, the support layer 11, and the layer 10 containing the carbon monoatomic layer substance are the same as those described in the section “Manufacturing Method for Three-Dimensional Structure”.

The laminated body according to the present aspect may be used to manufacture the three-dimensional structure of the embodiment described above.

### EXAMPLES

Hereinafter, the present disclosure will be described in more detail using specific examples. Note that; however, the present disclosure is not limited to the examples described below in any way.

### [Example 1] Production of Multilayer Film with Curve Induction Layer Being Constituted of Silk-Fibroin-Gel

A multilayer film 1 was produced according to the process of FIGS. 6(a) to 6(g). The multilayer film 1 was constituted in the same manner as the multilayer film 1a of FIG. 2A. In other words, a layer 10 containing a carbon monoatomic layer substance was single-layer graphene, and a curve induction layer 12 was hydrogel. Silk-fibroin-gel was used as hydrogel.

A glass substrate was used as a substrate 14. A sodium alginate solution was spin coated on the glass substrate, and then immersed in a calcium chloride solution so as to form a sacrifice layer 13 of calcium alginate gel.

Subsequently, the curve induction layer 12 was formed on the surface of the sacrifice layer 13. Silk-fibroin was dissolved in water and a silk-fibroin solution was adjusted. The silk-fibroin solution was spin coated onto the sacrifice layer 13 so as to form silk-fibroin-gel.

A support layer 11 was then formed on the surface of the curve induction layer 12. The support layer 11 constituted of poly-para-xylene (parylene) was formed by growing a para-xylene dimer by CVD on the curve induction layer 12. The thickness of the support layer 11 was 50 nm to 200 nm.

Next, single-layer graphene as the layer 10 containing the carbon monoatomic layer substance was transferred onto the support layer 11. Single-layer graphene was produced using CVD on a surface of copper foil, and the produced single-layer graphene was transferred to the surface of the support layer 11.

Subsequently, a photoresist was spin coated on the surface of the layer 10 containing the carbon monoatomic layer substance so as to form a resist layer 15. The resist layer 15 was irradiated with ultraviolet light via a photomask having an optional shape, so that a physical mask having an optional shape was patterned. Thereafter, the layer 10 containing the carbon monoatomic layer substance, the support layer 11, the curve induction layer 12, and the sacrifice layer 13 were etched by oxygen plasma. The etching was performed until reaching the sacrifice layer 13 deposited on the substrate 14. Finally, the resist layer 15 was removed by acetone.

### [Example 2] Production of Multilayer Film Using Multilayer Graphene as Curve Induction Layer

Similar to Example 1, the multilayer film 1 was produced according to the process of FIGS. 6(a) to 6(g). The multilayer film 1 was constituted in the same manner as the multilayer film 1b of FIG. 2B. In other words, the layer 10 containing the carbon monoatomic layer substance was single-layer graphene, and the curve induction layer 12 was multilayer graphene.

A calcium alginate gel layer was formed as the sacrifice layer 13 on a glass substrate selected as the substrate 14 by the same method as that in Example 1.

Subsequently, multilayer graphene was transferred as the curve induction layer 12 to the surface of the sacrifice layer 13. A plurality of pieces of single-layer graphene were laminated on the surface of the sacrifice layer 13 by repeating the operation of transferring the single-layer graphene described in the step of forming the layer 10 containing the carbon monoatomic layer substance in Example 1. The number of layers of the multilayer graphene was two.
Next, as in Example 1, poly-para-xylene was deposited as the support layer 11.
Subsequently, as in Example 1, single-layer graphene was transferred as the layer 10 containing the carbon monoatomic layer substance to the surface of the support layer 11.
Finally, as in Example 1, the photolithography technique was used to etch the multilayer film 1 and the sacrifice layer 13 so as to form an optional physical pattern.

### [Example 3] Production of Multilayer Film Using Graphene Oxide Flake as Layer Containing Carbon Monoatomic Layer Substance

Similar to Example 1, the multilayer film 1 was produced according to the process of FIGS. 6(a) to 6(g). The multilayer film 1 was constituted in the same manner as the multilayer film 1c of FIG. 2C. In other words, the layer 10 containing the carbon monoatomic layer substance was a flake of a graphene derivative, and the curve induction layer 12 was single-layer graphene. Graphene oxide was used as the graphene derivative.

A calcium alginate gel layer was formed as the sacrifice layer 13 on a glass substrate selected as the substrate 14 by the same method as that in Example 1.

Then, single-layer graphene was transferred as the curve induction layer 12 to the surface of the sacrifice layer 13 by the same method as that in the step of forming the layer 10 containing the carbon monoatomic layer substance in Example 1.
Next, as in Example 1, poly-para-xylene was deposited as the support layer 11.
Subsequently, a graphene oxide flake was applied to the surface of the support layer 11 to form the layer 10 containing the carbon monoatomic layer substance.
Finally, as in Example 1, the photolithography technique was used to etch the multilayer film 1 and the sacrifice layer 13 so as to form an optional physical pattern.

### [Example 4] Induction of Spontaneous Curvature of Multilayer Film

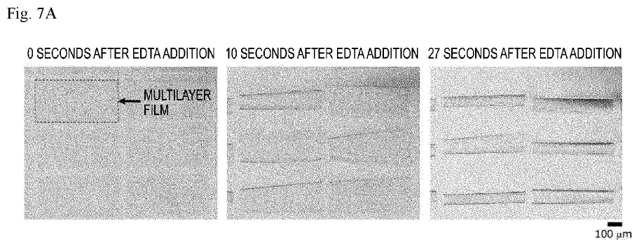

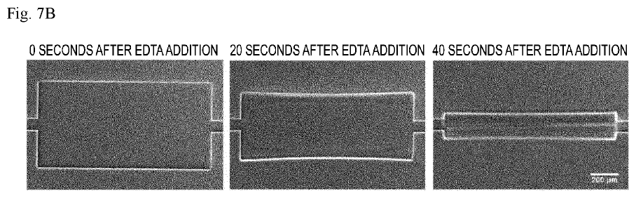

It is possible to induce the curvature of each of the multilayer films produced in the processes of Examples 1 to 3 by dissolving the sacrifice layer 13 constituted of calcium alginate gel by using EDTA. When the sacrifice layer 13 was dissolved by EDTA, in each of Examples 1 to 3, the rectangular multilayer film formed a cylindric structure as a representative curved structure. Images before and after the curvature of the multilayer film produced in Example 2 are shown in FIG. 7A. Images before and after the curvature of the multilayer film produced in Example 3 are shown in FIG. 7B.
As shown in FIG. 7B, when an elongated rectangle is connected as a hinge to a rectangular pattern for forming a cylindric structure, the flow of the curved multilayer thin film may be prevented. As a result, microscopic observation with the same field of view is facilitated.

### [Example 5] Effect of Curvature Radius by Number of Layers of Single-Layer Graphene

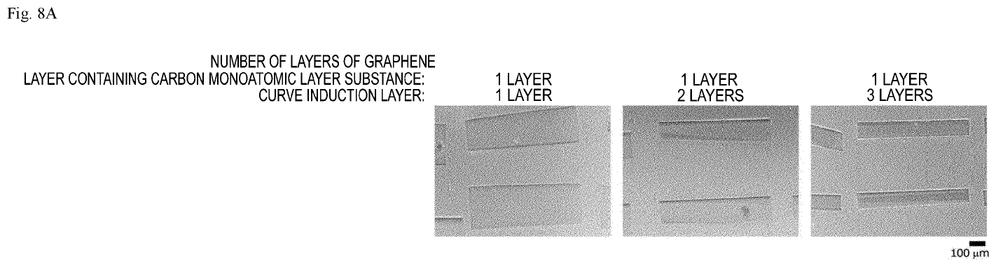

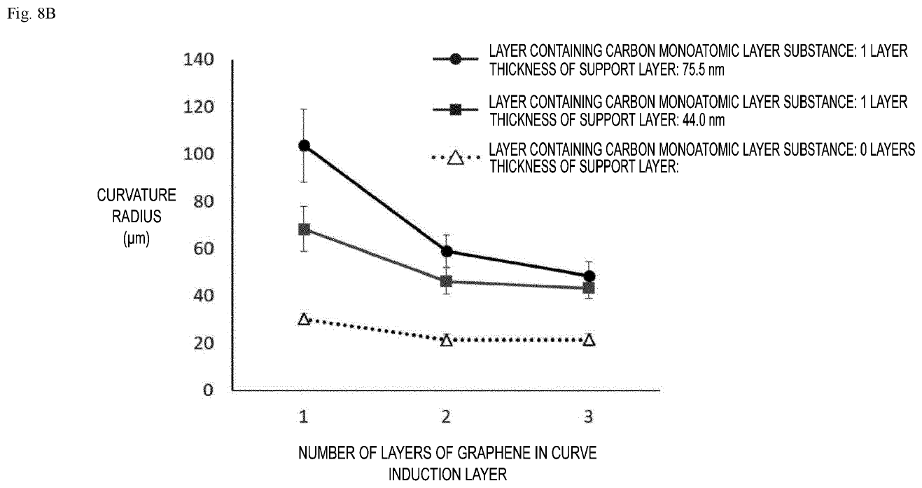

The curvature radius of the three-dimensional structure is determined by the thicknesses of the curve induction layer 12 and the support layer 11. In this example, the curve induction layer 12 was graphene, and the number of layers of the graphene was varied. The support layer 11 was a poly-para-xylene layer, and the layer 10 containing the carbon monoatomic layer substance was single-layer graphene. The thickness of the support layer 11 was 107.5 cm in the three-dimensional structure of FIG. 8A, and was 75.5 cm, 44.0 cm, or 112.8 cm in the three-dimensional structure of FIG. 8B. The multilayer film 1 was formed according to the process of FIGS. 6(a) to 6(e). Then, the multilayer film was cut into a rectangle of 300 μm×600 μm by using the photolithography technique. Subsequently, the sacrifice layer 13 was dissolved by EDTA to induce the curvature of the multilayer film 1.

FIG. 8A is a phase-contrast microscopic image of a three-dimensional structure. It was confirmed that the curvature radius of the three-dimensional structure decreased as the number of layers of graphene in the curve induction layer 12 increased.

FIG. 8B is a graph indicating a relationship among the number of layers of graphene in the curve induction layer 12, the thickness of the support layer 11, and the curvature radius of the three-dimensional structure. It was also confirmed in FIG. 8B that the curvature radius of the three-dimensional structure decreased as the number of layers of graphene in the curve induction layer 12 increased. In addition, it was confirmed that the curvature radius of the three-dimensional structure also decreased when the thickness of the support layer 11 increased.

### [Example 6] Enclosure of Cells by Multilayer Film

Culture cells were enclosed in the interior space of the three-dimensional structure according to the process of FIGS. 6(h) to 6(j). The multilayer film 1 was constituted in the same manner as the multilayer film 1b of FIG. 2B. In other words, the layer 10 containing the carbon monoatomic layer substance was single-layer graphene, and the curve induction layer 12 was multilayer graphene. The support layer 11 was a poly-para-xylene layer.

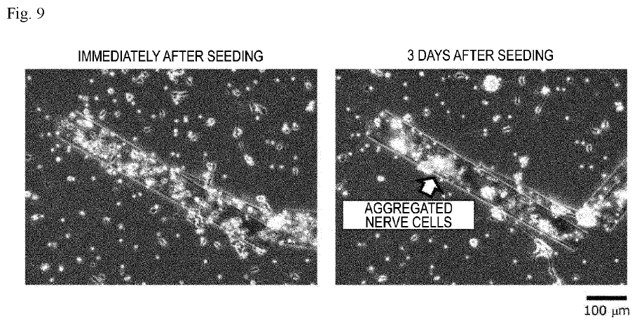

Primary-cultured hippocampus nerve cells harvested from a rat fetus were seeded on the multilayer film, and right after that, EDTA was added to dissolve the sacrifice layer 13 constituted of calcium alginate gel. As a result, the nerve cells engrafted on the layer containing the carbon monoatomic layer substance were enclosed in the cylindric three-dimensional structure. FIG. 9 shows microscopic images of the cells in the same cylindric structure on the first and third days of the culture. Migration of the nerve cells to the outside is suppressed by the curved structure. Thus, as shown in FIG. 9, the nerve cells may be cultured while maintaining the contact with the layer containing the carbon monoatomic layer substance in contact with the interior space of the cylindric three-dimensional structure. Further, it is possible to bring graphene into contact with three-dimensionally multiplied nerve cells inside the curved structure of the three-dimensional structure.

### INDUSTRIAL APPLICABILITY

According to the present disclosure, there are provided a three-dimensional structure having a three-dimensionally curved structure in which a layer containing a carbon monoatomic layer substance such as a carbon monoatomic layer is set on the inner side, and a manufacturing method for the three-dimensional structure. Because the layer containing the carbon monoatomic layer substance is in contact with the interior space of the three-dimensional structure, the three-dimensional structure is able to detect electric characteristics of the interior space with high sensitivity. In the case where cells are present in the interior space, because it is possible to bring the cells into contact with the layer containing the carbon monoatomic layer substance, the present disclosure is applicable to the detection of action potentials or the like of the cells and the detection of released biomolecules by the cells.

### REFERENCE SIGNS LIST

1 Multilayer film
2 Cell
4 Insulating layer
5a, 5b Probe
6 Biomolecule detection probe
10 Layer containing a carbon monoatomic layer substance
11 Support layer
12 Curve induction layer
13 Sacrifice layer
14 Substrate
15 Resist layer
20 Single-layer graphene
21 Multilayer graphene
22 Flake of a graphene derivative
23 Hydrogel
30 Conductor connected to a layer 10 containing a carbon monoatomic layer substance
31 Conductor connected to a curve induction layer 11
60 Signal molecule
61 Specific binding substance
100, 100′ Three-dimensional structure
200, 201 Electric signal detection device
S Interior space of a three-dimensional structure

1. A three-dimensional structure comprising
a multilayer film that is made into three-dimensionally curving to form an interior space of the three-dimensional structure,
wherein the multilayer film includes a layer containing a carbon monoatomic layer substance, a support layer, and a curve induction layer configured to induce the three-dimensionally curving,
the layer containing the carbon monoatomic layer substance is in contact with the interior space, and the support layer is positioned between the layer containing the carbon monoatomic layer substance and the curve induction layer.

2. The three-dimensional structure according to claim 1,
wherein the carbon monoatomic layer substance is graphene or a derivative of the graphene.

3. The three-dimensional structure according to claim 1,
wherein a cell is present in the interior space.

4. The three-dimensional structure according to claim 3,
wherein the layer containing the carbon monoatomic layer substance is in contact with the cell.

5. The three-dimensional structure according to claim 1,
wherein the layer containing the carbon monoatomic layer substance includes a biomolecule detection probe.

6. An electric signal detection device comprising:
the three-dimensional structure according to claim 1; and
a conductor connected to the layer containing the carbon monoatomic layer substance.

7. A manufacturing method for a three-dimensional structure, the method comprising:
(a) forming a multilayer film that includes a layer containing a carbon monoatomic layer substance, a support layer, and a curve induction layer configured to induce a curved structure, the support layer being positioned between the layer containing the carbon monoatomic layer substance and the curve induction layer; and
(b) causing the multilayer film to form, in a self-organized manner, a three-dimensionally curved shape where the layer containing the carbon monoatomic layer substance is set on an inner side of the three-dimensionally curved shape by utilizing a gradient of strain in a thickness direction of the multilayer film as a drive force.

8. A laminated body comprising:
a substrate;
a sacrifice layer laminated on the substrate;
a curve induction layer laminated on the sacrifice layer and configured to induce a curved structure;
a support layer laminated on the curve induction layer; and
a layer containing a carbon monoatomic layer substance laminated on the support layer.
